(19) United States
(12) Patent Application Publication
(10) Pub. No.: US 2025/0273325 A1
(43) Pub. Date: Aug. 28, 2025

(54) OPHTHALMIC SYSTEM AND TERMINAL DEVICE

(71) Applicant: TOPCON CORPORATION, Tokyo (JP)

(72) Inventor: Noriyasu TAKEDA, Tokyo (JP)

(73) Assignee: TOPCON CORPORATION, Tokyo (JP)

(21) Appl. No.: 19/065,333

(22) Filed: Feb. 27, 2025

(30) Foreign Application Priority Data
Feb. 28, 2024 (JP) 2024-028797

(51) Int. Cl. G16H 30/40; A61B 3/00; G06T 11/20; G16H 10/60; G16H 40/60

(52) U.S. Cl. CPC G16H 30/40; A61B 3/0058; G06T 11/206; G16H 10/60; G16H 40/60; G06T 2210/41

(57) ABSTRACT

An ophthalmic system includes a server including a memory storing a result of an examination or an image diagnosis related to an eyeball of a subject and a terminal device connected to the server. The terminal device includes a display displaying the result and a display controller controlling a display content and a display mode. The memory stores the result in association with device information including a manufacturer or a model name of an ophthalmic device used at that time. The display displays the result in a time-series graph. When a change in the manufacturer or the model name from that in a previous examination is detected based on the device information, the display controller sets the display mode of the graph to a first change mode.

### CROSS-REFERENCE TO RELATED APPLICATION

This application is based on and claims priority under 35 USC 119 from Japanese Patent Application No. 2024-028797 filed on Feb. 28, 2024, the entire content of which is incorporated herein by reference.

### TECHNICAL FIELD

The present disclosure relates to an ophthalmic system and a terminal device.

### BACKGROUND

A related art discloses a management system that centrally manages medical examination results, and helps examining doctors to monitor the progress observation of ophthalmic diseases by using numerical values such as an intraocular pressure value and an eye refractive power, and images such as a fundus image, an optical coherence tomography (OCT) image. An examining doctor can confirm the results from a terminal device of the system. Such a management system has come to have a prediction function that estimates numerical values in the future based on past examination results.

Patent Literature (JP 6489193 B) discloses an ophthalmic analysis device that obtains an analysis result on subject eye's tomographic images by using Optical Coherence Tomography (OCT images) that have been acquired on different dates, and provides statistical information generated based on time-series data of the analysis result.

The analysis result includes a result of a fundus tomographic image and an anterior-segment tomographic image. Additionally, the analysis result may include a thickness of the subject eye (such as, a corneal thickness, a lens thickness, a thickness of at least one layer of a retinal layer and a choroid) and a curvature of the subject eye (such as, a corneal curvature, a lens anterior surface/posterior surface curvature, a curvature of at least one layer of a retinal layer).

Furthermore, the analysis device is configured to acquire regression lines by regression analysis on the time-series data and provide trend graph (prediction graph) charts using the regression lines (see Paragraphs 0011 to 0013, and 0022 and FIG. 5 of JP 6489193 B).

### SUMMARY

However, the ophthalmic analysis device disclosed in JP 6489193 B has the following problems.

When a subject patient undergoes a medical examination at a different medical institution that the subject patient usually presents to, the examination may be conducted through different devices of different manufacturers that the subject patient usually uses. Then, if an examining doctor (such as, an ophthalmologist, an optometrist, and an orthoptist) undoubtedly accepts the numerical values of the examination as they are, there may have a problem that eye disease is overlooked due to a difference in a numerical value criterion or an error depending on the device. Furthermore, it is highly likely that the trend graph charts must provide an inaccurate result.

The present disclosure has been made in view of such circumstances, and an object thereof is to provide an ophthalmic system that allows an examiner to correctly evaluate an examination result even when a device manufacturer, a model, or the like used in the examination is different from the one usually used.

An ophthalmic system includes a server including a memory storing a result of an examination or an image diagnosis related to an eyeball of a subject and a terminal device connected to the server. The terminal device includes a display displaying the result of the examination or the image diagnosis and a display controller configured to control a display content and a display mode. The memory stores the result of the examination or the image diagnosis in association with device information including a manufacturer or a model name of an ophthalmic device used at that time. The display displays the result of the examination or the image diagnosis in a time-series graph. In a case where a change in the manufacturer or the model name from that in a previous examination is detected based on the device information, the display controller sets the display mode of the graph to a first change mode different from a normal mode without the change.

According to the present disclosure, an examiner can correctly evaluate an examination result even when a device manufacturer, a model, or the like used in the examination is changed.

### BRIEF DESCRIPTION OF DRAWINGS

FIG. 1 is an overall diagram of an ophthalmic system of the present disclosure.

FIG. 2 is a diagram illustrating details of each component of the ophthalmic system.

FIG. 3 is a diagram showing a screen example of a terminal device (display unit).

FIG. 4 is a diagram showing a screen example (progress graph) of the terminal device (display unit).

FIG. 5A is a diagram illustrating a progress graph of an intraocular pressure value of a certain subject.

FIG. 5B is a diagram illustrating a progress graph (a pop-up window of a comparison table) of the intraocular pressure value of a certain subject.

FIG. 6A is a diagram illustrating a progress graph (a predicted value display) of the intraocular pressure value of a certain subject.

FIG. 6B is a diagram illustrating a progress graph (a pop-up window related to prediction) of the intraocular pressure value of a certain subject.

### DETAILED DESCRIPTION

Hereinafter, an example of the present disclosure will be described with reference to

the drawings, but the scope of the present disclosure is not limited to illustrative embodiments described here, and various change modes can be made without departing from the gist of the present disclosure. Further, when a plurality of upper limit values and lower limit values are described as specific parameters, any upper limit value and any lower limit value among the upper limit values and the lower limit values can be combined to obtain a suitable numerical value range.

### First Illustrative Embodiment

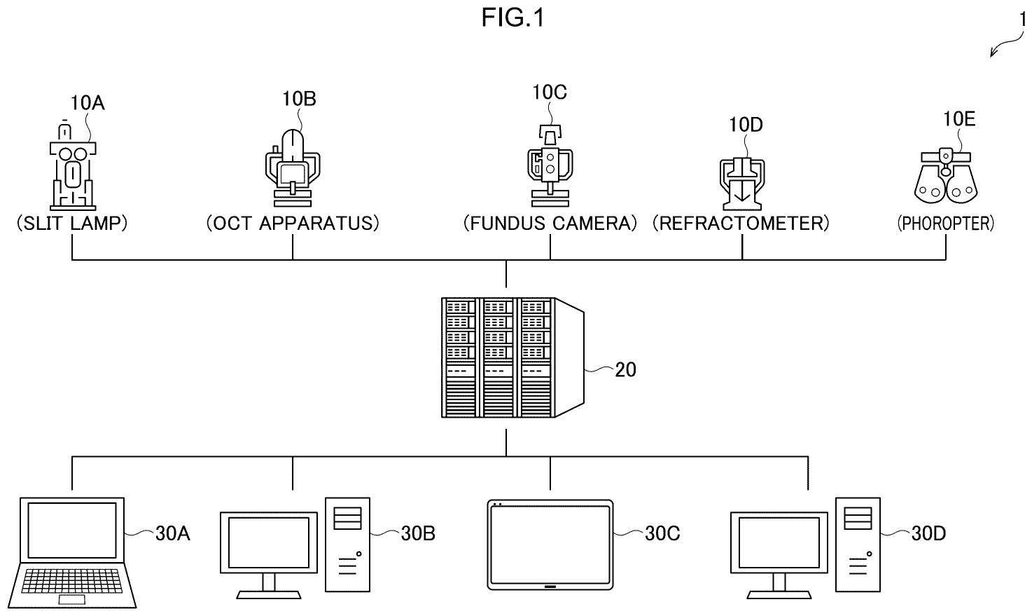

FIG. 1 is an overall diagram of an ophthalmic system 1 according to a first illustrative embodiment of the present disclosure. The ophthalmic system 1 includes a plurality of ophthalmic devices 10 (10A to 10E), a server device 20, and a plurality of terminal devices 30 (30A to 30D). The ophthalmic devices 10 (or a communication device connected to the ophthalmic device 10) and the server device 20 are connected via a network, and the terminal devices 30 and the server device 20 are connected via a network. For example, an examination result obtained by each of the ophthalmic devices 10 is stored into the server device 20 via the network. Therefore, an examiner using the terminal device 30 can confirm the examination result. The number of ophthalmic devices 10 and the number of terminal devices 30 are merely examples, and are not limited.

As shown in FIG. 1, the ophthalmic devices 10 include a slit lamp 10A, an OCT apparatus 10B, a fundus camera 10C, a refractometer 10D, and a phoropter 10E. Each of the ophthalmic devices 10 may be owned by different medical institutions (a general hospital, an ophthalmic clinic, a university hospital, or the like), or a specific medical institution may own a plurality of the devices.

For example, the fundus camera 10C is a device owned by many medical institutions, but the medical institutions introduce models having different manufacturers and functions. However, the ophthalmic system 1 allows the examiner to confirm the examination result, including information obtained by another medical institution, on the terminal devices 30 regardless of the manufacturer, the model, or the like.

Here, features of each ophthalmic device 10 will be briefly described. The slit lamp

(slit lamp microscope) 10A is a device that acquires an image of a cross-section of a cornea (anterior segment image) by irradiating a subject eye, which is an eyeball of a subject, with elongated slit-shaped illumination light from an oblique direction. The slit lamp 10A is used to examine various anterior segments such as corneal shapes, corneal endothelial cells, and Meibomian glands.

The OCT apparatus 10B is an apparatus that acquires a tomographic image of a fundus (retina) by dividing low-coherence light into two parts, irradiating the fundus with one part, causing backscattered light of the one part to interfere with the other part, and receiving the light with a CCD image sensor or the like. The tomographic image is used to determine eye diseases such as glaucoma, diabetic retinopathy, and retinal pigment degeneration. As the OCT apparatus 10B, a device of any type such as a Fourier domain type, a time domain type, or a swept source type can be used.

The fundus camera 10C is a device that irradiates a fundus portion of the subject eye with illumination light and receives fundus reflected light by a light receiving element such as charge coupled devices (CCD) image sensor to acquire data of a fundus image. The fundus image is used to determine eye diseases such as glaucoma, diabetic retinopathy, and age-related macular degeneration.

The refractometer 10D is an examination device capable of measuring various eye characteristics such as an intraocular pressure value, an eye refractive power, and a corneal curvature of the subject eye. Further, the phoropter (vision tester) 10E is an examination device used to select a lens suitable for the subject eye. The phoropter 10E includes a plurality of lenses, and by inserting one or a plurality of the lenses on the optical axis, the eye refractive power such as myopia, hyperopia, and astigmatism, or a binocular visual function can be measured.

The ophthalmic device 10 may include devices other than those described above. An example is a scanning laser ophthalmoscope which performs scanning using a light beam to irradiate the fundus and receives reflected light or fluorescence at a predetermined portion with a CCD or the like to acquire image data of the fundus image. Another example is a specular microscope capable of imaging the corneal endothelial cells, measuring a density, and measuring the corneal thickness.

The server device 20 includes a storage unit therein, and stores the examination result, the image data, and the like of the ophthalmic devices 10 transmitted via the network. Further, since the terminal devices 30 are connected to the server device 20, the terminal devices 30 can always access the examination result and the like stored in the storage unit of the server device 20. A type of the server device 20 is not particularly limited, and may be, for example, a cloud server.

The terminal devices 30 (30A to 30D) are each a PC, a notebook PC, a tablet terminal, or the like provided in each medical institution. Software for using the ophthalmic system 1 is installed in each of the terminal devices 30. Therefore, the examiner can confirm, on the terminal devices 30, the examination result and the image data stored in the server device 20. Further, since the examiner can confirm recorded items of an electronic medical record in addition to the examination results and the like acquired by other medical institutions, the examiner determines the presence or absence of a disease of the subject eye in consideration of each piece of information.

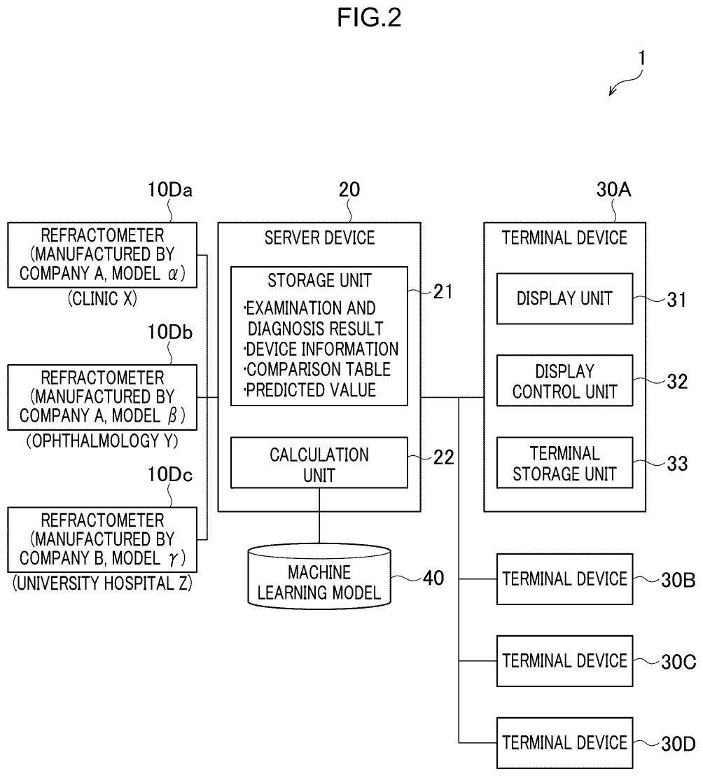

FIG. 2 is a diagram illustrating each configuration of the ophthalmic system 1 of the present disclosure. Hereinafter, internal configurations of the ophthalmic devices 10, the server device 20, and the terminal devices 30 will be described in detail.

FIG. 2 illustrates three types of refractometers 10Da, 10Db, and 10Dc as the plurality of ophthalmic devices 10. The refractometer 10Da is of model a manufactured by company A, and is a device installed in clinic X. The refractometer 10 Db is of model β manufactured by company A, and is a device installed in ophthalmology Y. The refractometer 10Dc is of model γ manufactured by company B, and is a device installed in university hospital Z.

As shown in the figure, even in the refractometer 10D capable of performing the same examination, the manufacturer may be different depending on the medical institution, or the model and the model number may be different even if the manufacturer is the same. Therefore, it is assumed that the numerical values of the examination results obtained by the respective devices slightly differ depending on the device used in the examination. Since there are many different manufacturers and models of the OCT apparatus 10B, the numerical values of the thickness of each of the layers constituting the retina differ.

Next, the server device 20 includes therein a storage unit 21 and a calculation unit 22. The storage unit 21 stores examination and diagnosis results (the examination results and the image data) obtained by the ophthalmic devices 10. The storage unit 21 also stores device information such as the manufacturer and model of the ophthalmic device 10 used in the examination. The examination and diagnosis result and the device information are stored in association with each other. The storage unit 21 is a storage medium such as a semiconductor memory, an optical disk, or a magnetic disk in which data can be written.

For example, when a subject undergoes an ophthalmic examination and diagnosis in

clinic X, an examination and diagnosis result of the subject and device information indicating that the refractometer 10Da of model a manufactured by company A is used are stored in association with each other. The device information may be, for example, a symbol or a number such as “Aα”. Although details will be described later, a display control unit (which may also be referred to as a display controller) 32 of the terminal device 30 recognizes the device information and changes a display mode in a time-series graph (a progress graph).

The storage unit 21 stores a comparison table. The comparison table includes information on a numerical tendency for each examination item of each manufacturer. As an example of the comparison table, the refractometers 10Da and 10 Db manufactured by company A and the refractometer 10Dc manufactured by company B are compared, and information such as a tendency that a higher numerical value tends to be given and an allowable range of an error is formed into a chart. The comparison table may be information on a numerical tendency for each examination item of each model. By referring to the comparison table, the examiner can make a determination in consideration of characteristics of the ophthalmic device 10 used in the examination.

The calculation unit (may also be referred to as a calculator) 22 is a processor (a CPU, a GPU, an FPGA, or the like) capable of calculating a predicted value of a predetermined examination item based on a result of an examination or an image diagnosis of the subject with reference to a machine learning model 40 to be described later. Since the predicted value is displayed in a progress graph of the intraocular pressure value displayed on the terminal device 30, the predicted value is useful when the examiner reports, advises, or warns a recovery state of the eye disease to a patient. The created predicted value is stored in the storage unit 21.

Here, the machine learning model 40 is a database connected to the server device 20 and subjected to machine learning to output the predicted value. The machine learning model 40 is obtained as a result of machine learning in which many results of the examination or the image diagnosis for each examination item are input as training data and the predicted value is output for each examination item.

For example, the machine learning is performed by inputting measurement values of the intraocular pressure values of many subjects to any computer as the training data and adding an examination result of the glaucoma or the like as necessary as training data. By using the obtained machine learning model 40, it is possible to output information such as a predicted value of a future intraocular pressure value, a transition of the numerical value, and an incidence rate of the glaucoma. A method of the machine learning is not particularly limited, and various methods such as unsupervised learning and deep learning can be adopted. The machine learning model 40 may be built in the server device 20.

Each of the terminal devices 30A to 30D includes therein a display unit 31, the display control unit 32, and a terminal storage unit 33. Although an example of the terminal device 30A will be described below, the terminal devices 30B to 30D also have similar basic configurations and functions.

The display unit 31 is a display that displays the result of the examination or the image diagnosis. On the display unit 31, the examiner can confirm the device information, the comparison table, the recorded items of the electronic medical record recorded in another medical institution, and the like in addition to the examination and diagnosis result. A type of the display may be any type such as liquid crystal, plasma, or organic EL, and may be a touch panel type.

The display control unit 32 is a processor that controls a display content and a display mode of the display unit 31. Although details will be described later, the display control unit 32 changes the display mode of the progress graph on the display unit 31 or causes a pop-up window to appear when a predetermined condition is satisfied.

The terminal storage unit 33 is a storage medium such as a semiconductor memory, an optical disk, or a magnetic disk in which data can be written. Since the above-described comparison table needs to be updated as appropriate, for example, when a new type of ophthalmic device 10 is added to the ophthalmic system 1, it is preferable that the comparison table is stored in the storage unit 21 of the server device 20 and always accessible from any terminal device 30. However, the comparison table may be downloaded by the examiner and stored in the terminal storage unit 33, and may be used when necessary.

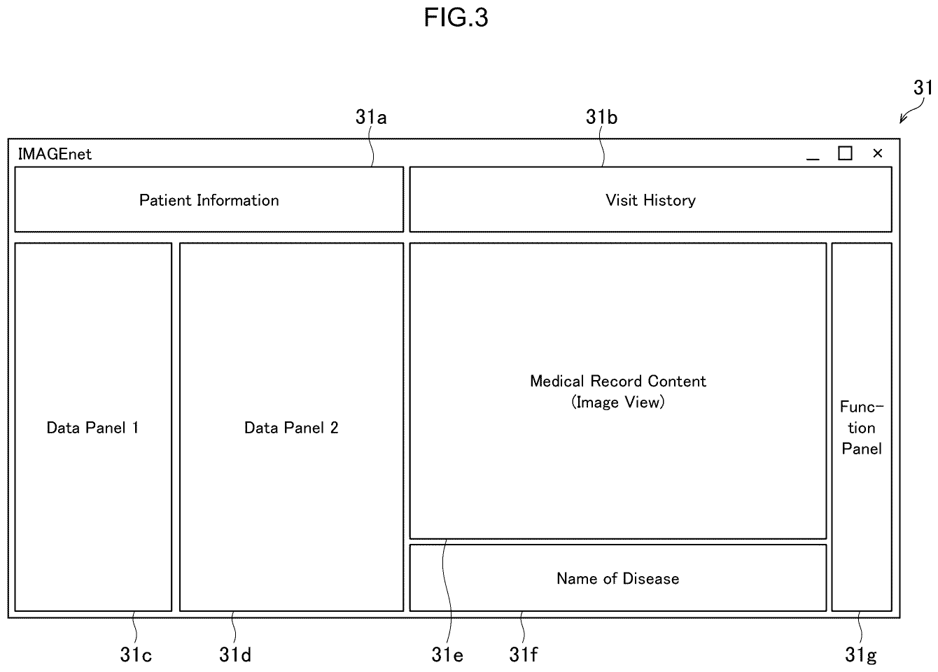

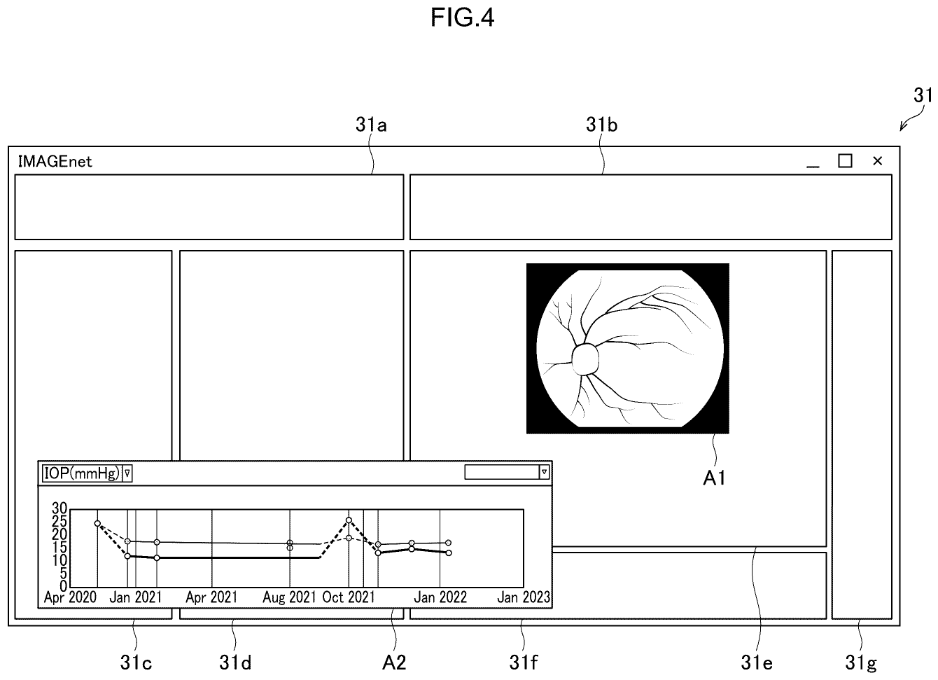

Next, a display example of the terminal device 30 (the display unit 31) will be described with reference to FIGS. 3 and 4.

FIG. 3 is a screen of the electronic medical record of the ophthalmic system 1 displayed on the display unit 31. A region 31a is a region of patient information (Patient Information), and information such as the name, age, sex, and address of the patient (the subject) is displayed therein. A region 31b is a region of hospital visit history (Visit History), and information such as a medical institution at which the patient receives the examination, a date and time, and a medicine prescribed at that time are displayed therein.

A region 31c is a data panel (Data Panel 1) on which an examination result is displayed when a certain examination date is designated. In the region 31c, for example, information such as a visual acuity, an intraocular pressure value, and a found disease obtained in the examination of the day is displayed. The mode may be a mode in which a surgery history of the patient is displayed.

A region 31d is also a data panel (Data Panel 2) on which the examination result is displayed, and the examination result other than that displayed in the region 31c is displayed.

In the region 31d, information such as the visual acuity and the intraocular pressure value of a day different from that of the region 31c may be displayed and compared, or other examination results of the same day as that of the region 31c may be displayed.

A region 31e is a region in which recorded items of the electronic medical record (Medical Record Content) are displayed. Since the region 31e is a relatively large region, a result of the image data (Image View) can also be displayed.

A region 31f is a region in which a name of the eye disease (Name of Disease) is displayed. A region 31g is a region of a function button panel (Function Panel) for performing various display switching. Since it may be desired to compare a plurality of pieces of image data or progress graphs side by side, the image data or the like may be displayed in the regions 31a to 31g in a superimposed manner.

FIG. 4 is a screen example when a fundus image A1 and a progress graph A2 are displayed on the electronic medical record of the ophthalmic system 1. When the fundus image Al of a certain examination date is displayed on the display unit 31, the display control unit 32 displays the fundus image Al in the region 31e.

Further, when a progress graph A2 of the intraocular pressure value including the examination date is displayed, the display control unit 32 superimposes the progress graph A2 on, for example, the regions 31c to 31f to allow the examiner to visually recognize both the fundus image Al and the progress graph A2. The progress graph A2 can also be moved by a mouse, a touch pen, or the like.

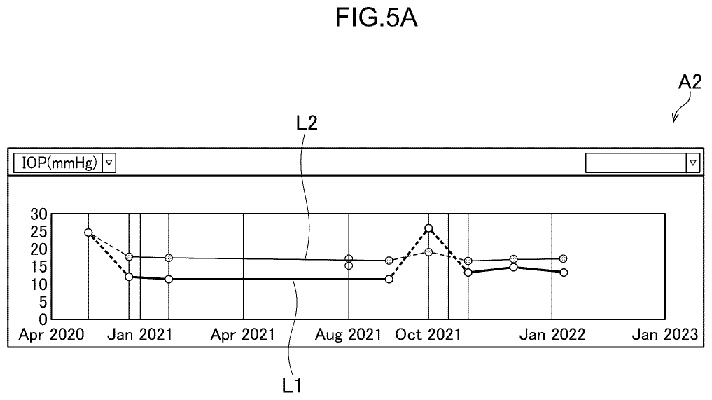

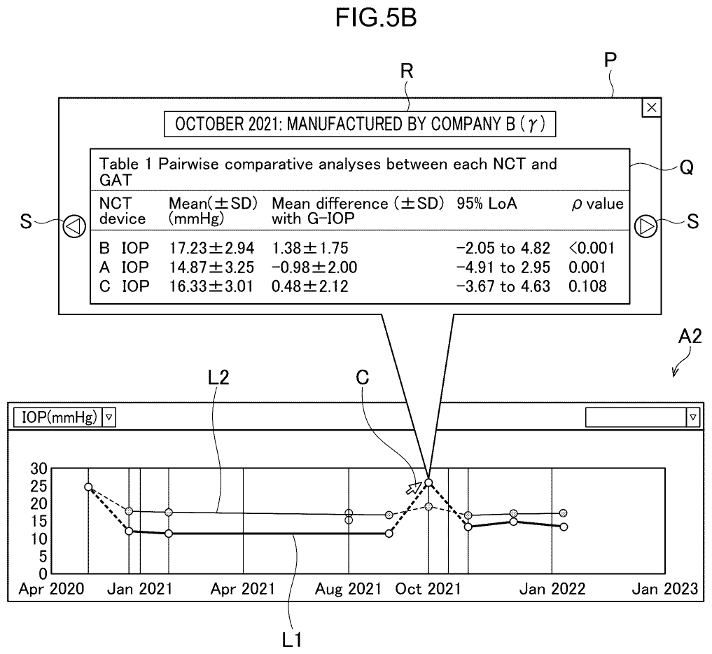

Next, the progress graph of the intraocular pressure value displayed on the display unit 31 will be described with reference to FIGS. 5A and 5B.

In the progress graph A2 of FIG. 5A, a horizontal axis represents time. A vertical axis represents an intraocular pressure value (IOP) (mmHg) obtained by the refractometer 10D. Here, a thick polygonal line L1 corresponds to the intraocular pressure value of a left eye, and a thin polygonal line L2 corresponds to the intraocular pressure value of a right eye. In addition to numerical values of a viewing angle, an axial length, and the like, a right vertical axis may be set to an axis representing the thickness of the retina (a central region membrane thickness, a nerve fiber layer thickness, a ganglion cell layer thickness, or the like) obtained by the OCT apparatus 10B, and a new graph may be added to the progress graph A2.

Hereinafter, the display mode will be described using the polygonal line L1, which is an intraocular pressure value graph of the left eye, as an example. First, in the intraocular pressure value graph, a solid line portion indicates a progress when the subject receives an examination at a medical institution (for example, clinic X) that the subject usually visits. Further, a broken line portion (an example of a “first change mode” of the present disclosure) indicates a progress when the subject receives an examination at a medical institution (for example, the university hospital Z) different from that the subject usually visits.

When the medical institution is different, the manufacturer or model of the ophthalmic device 10 is different, and thus a reference of the numerical value may change. Therefore, the terminal device 30 (the display control unit 32) refers to the above-described device information (for example, a symbol or a numerical value) and detects a change in the device from that at the time of a previous examination. Then, when the change is detected, the display control unit 32 changes the display mode of the intraocular pressure value graph to allow the examiner to recognize the change.

A circle in the intraocular pressure value graph indicates an actual examination date, and when the examiner designates (for example, clicks or places a cursor on) the circle, a specific numerical value or the like can be confirmed. FIG. 5B shows a state when the examiner designates a circle (October 2021) in the broken

line portion. When the examiner places a cursor C on the circle, a pop-up window P (a “window” of the present disclosure) appears in the display unit 31. A comparison table Q is shown in the pop-up window P. The comparison table Q is a list in which numerical tendencies among manufacturers are mainly compared. The examiner can also instruct a forward button S in the pop-up window P to display a comparison table and other information on a second page. That is, the information in the pop-up window P assists the examiner in making an accurate determination.

A device display R related to the ophthalmic device 10 used on the examination date is shown above the comparison table Q. When the data of the ophthalmic device 10 exists in the comparison table Q, a corresponding portion may be highlighted or a mark such as a star mark may be displayed at a head of the corresponding portion. Accordingly, the examiner can immediately grasp the characteristics of the ophthalmic device 10 used in the examination.

By displaying the pop-up window P, the examiner can acquire device characteristics of a device that gives a higher numerical value, a device that gives a lower numerical value, and the like. Then, the examiner can determine the presence or absence of an abnormality taking into consideration that the ophthalmic device 10 (in this example, the refractometer 10D) is changed.

In the example of the examination date (October 2021), it can be seen from the device display R that the intraocular pressure value was measured by a device of model y manufactured by company B. Further, referring to the comparison table Q, it is disclosed that the device manufactured by company B (IOP manufactured by company B) gives a higher mean value (Mean mmHg). Therefore, the examiner can determine that an actual increase width is small although the intraocular pressure value increases in the examination on this day. In this way, even when examinations are performed by different ophthalmic devices 10 in a community medical cooperation, the examiner can correctly evaluate the examination results.

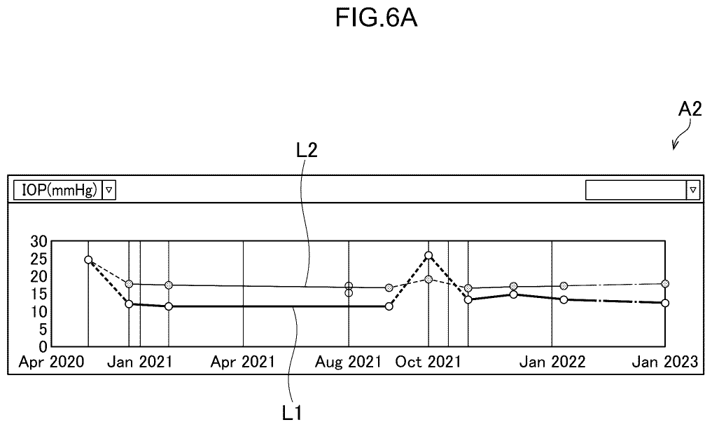

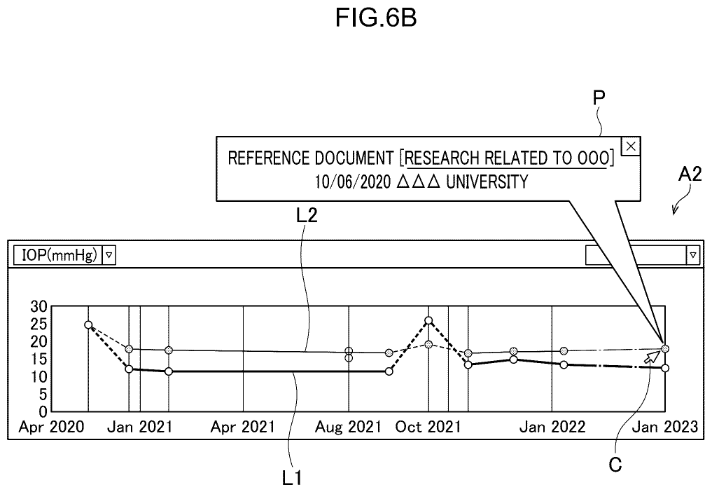

Next, display of the predicted value will be described with reference to FIGS. 6A and 6B.

As described above, the server device 20 (calculation unit 22) can predict a numerical value such as the intraocular pressure value of the subject using the machine learning model 40. Since the prediction is performed in response to a request from the terminal device 30 and the data is stored in the storage unit 21 of the server device 20, the examiner can access the prediction result from the terminal device 30.

For example, the examiner can instruct execution of the prediction and display of the predicted value from a function button panel (the region 31g) of the display unit 31. Then, as shown in FIG. 6A, the display control unit 32 shows the predicted value of the intraocular pressure value graph by a one-dot chain line (an example of a “second change mode” of the present disclosure) to allow the examiner to recognize the predicted value.

The predicted value is added to the progress graph A2 in a mode different from a normal mode (solid line) and a mode (broken line) when the ophthalmic device 10 is changed. Therefore, the examiner can easily read the predicted value from the progress graph A2. Then, the examiner can give advices or warnings to the subject based on the predicted value. A function of displaying both predicted values when treatment is continued with medicine or the like and when treatment is left may be provided.

The prediction of various numerical values such as the intraocular pressure value may be calculated based on a result of a paper or a conference presentation. In such a case, when the examiner designates a circle (January 2023) at an end portion of the one-dot chain line with the cursor C, a pop-up window P appears in the display unit 31 as shown in FIG. 6B. In the pop-up window P, a title of a related paper or conference presentation is presented.

As described above, the information of the pop-up window P indicates a basis of a current predicted value to the examiner. A name of a reference document or the like may be shown, and further a function of jumping to a document on the Internet, a paper, or a record of an academic conference may be provided by a link function.

### Second Illustrative Embodiment

Next, the terminal device 30 according to a second illustrative embodiment of the present disclosure will be described.

The terminal device 30 is the same as in the first illustrative embodiment in that the

terminal device 30 includes the display unit 31, the display control unit 32, and the terminal storage unit 33 (see FIG. 2). For example, it is sufficient for the terminal device 30 owned by clinic X that information of a plurality of patients (subjects) visiting the hospital can be viewed thereon basically. Therefore, the examiner can periodically download the examination and diagnosis results and the like of the patients from the server device 20 and appropriately use the results stored in the terminal storage unit 33.

In such a case, the terminal device 30 (the display unit 31) can also display a result of an examination or an image diagnosis of a patient as a time-series progress graph. Further, the display control unit 32 detects a change in the device from a previous examination based on device information (a symbol or a number) including a manufacturer or a model name of the ophthalmic device at the time of examination of the patient. When the change is detected, the display control unit 32 changes a display mode of the progress graph (polygonal line) (from a solid line to a broken line) to allow the examiner to recognize the change.

The comparison table Q (see FIG. 5B) regarding the numerical tendency for each examination item of each manufacturer may also be periodically downloaded, stored in the terminal storage unit 33, and appropriately used. When any examination date (circle) in the progress graph is designated, the display control unit 32 causes a pop-up window P (see FIG. 5B) including at least the comparison table Q to appear. As for the examination and diagnosis result, the device information (the device display R), and the like, those stored in the terminal storage unit 33 may be called and displayed on the display unit 31.

With the comparison table Q and the device display R of the pop-up window P (see FIG. 5B), the examiner can immediately grasp the characteristics of the ophthalmic device 10 used in the examination. Further, the examiner can acquire device characteristics of a device that gives a higher numerical value, a device that gives a lower numerical value, and the like. Accordingly, the examiner determines the presence or absence of an abnormality taking into consideration that the ophthalmic device 10 is changed, and thus a current examination result can be correctly evaluated.

The present disclosure is not limited to the illustrative embodiment described above and can be implemented in various modes without departing from the gist thereof.

In the illustrative embodiment described above, the pop-up window P including the comparison table Q and the like appears in the progress graph A2, but the present invention is not limited thereto. For example, a list including an examination date and examination numerical values (an intraocular pressure value, eye refractive power, visual acuity, and the like) may be displayed on the pop-up window P.

When the list is displayed, a change in the ophthalmic device 10 from the previous examination may be detected. At this time, the display control unit 32 may cause the examiner to recognize the change by coloring or marking a cell or value of at least one of the examination date and the numerical values, or underlining the numerical values. Further, when the predicted value is displayed in the list, the predicted value may be displayed in another mode such as showing a cell or a numerical value in a color different from that for the change in the ophthalmic device 10.

### Appendix

At least the following contents are disclosed by the above description.

(1) An ophthalmic system including:
a server including a memory storing a result of an examination or an image diagnosis related to an eyeball of a subject; and
a terminal device connected to the server, the terminal device including:
   a display displaying the result of the examination or the image diagnosis; and
   a display controller configured to control a display content and a display mode,
wherein the memory stores the result of the examination or the image diagnosis in association with device information including a manufacturer or a model name of an ophthalmic device used at that time,
wherein the display displays the result of the examination or the image diagnosis in a time-series graph, and
wherein in a case where a change in the manufacturer or the model name from that in a previous examination is detected based on the device information, the display controller sets the display mode of the graph to a first change mode different from a normal mode without the change.

The present disclosure is an ophthalmic system in which the terminal device such as a PC or a tablet terminal is connected to the server. The result of the ophthalmic examination or image diagnosis is associated with the device information including the manufacturer or model name of the ophthalmic device used in the examination, and is stored in the memory of the server. Since the result is displayed on the display of the terminal device, an examiner can confirm the result.

Further, since the display displays the result of the examination or the image diagnosis in a time-series graph, the examiner can confirm a transition of a numerical value or an image. For example, the manufacturer of the ophthalmic device may be changed from that of the previous examination, but the change can be detected because the device information is stored. At this time, the display controller sets the display of the graph to the first change mode different from the normal mode. Accordingly, the examiner determines the presence or absence of an abnormality taking into consideration that the ophthalmic device is changed, and thus a current examination result can be correctly evaluated.

(2) The ophthalmic system according to (1),
wherein the memory stores a comparison table related to a numerical tendency for each examination item of each of the manufacturers, and
wherein the display controller causes a window including at least the comparison table to appear when any examination time in the graph is designated.

The memory of the server stores the comparison table related to the numerical tendency for each examination item of each manufacturer, and contents of the comparison table are displayed on the display of the terminal device. In particular, when a predetermined examination date in the graph is designated, the display controller causes the window to appear on the display and displays a comparison table of a related examination item. Accordingly, the examiner can acquire device characteristics of a device that gives a higher numerical value, a device that gives a lower numerical value, and the like.

(3) The ophthalmic system according to (2),
wherein in the window, device display is performed in which the manufacturer or the model name of the ophthalmic device used in the examination is recognizable.

According to the configuration, the comparison table is displayed in the window, and the device display in which the manufacturer of the ophthalmic device used in the examination can be recognized is performed. When information of the used ophthalmic device is contained in the comparison table, the information may be indicated by a mark or the like. Accordingly, the examiner can immediately grasp characteristics (giving a higher numerical value or the like) of the ophthalmic device used in the examination.

(4) The ophthalmic system according to (1), further including:
a machine learning model connected to the server and configured to be subjected to machine learning such that many results of the examination or the image diagnosis for each examination item are received as training data, and a predicted value for a corresponding one of the examination items is output,
wherein the server includes a calculator configured to refer to the machine learning model to calculate the predicted value of a predetermined one of the examination items based on the result of the examination or the image diagnosis of the subject, and
wherein the display controller sets the display mode of the graph to a second change mode different from the normal mode and the first change mode when displaying the predicted value of the predetermined examination item.

In the present disclosure, the machine learning model subjected to machine learning to output the predicted value for each examination item is prepared in advance and can be used from the server. Further, the calculator of the server can refer to the machine learning model to calculate the predicted value of a predetermined examination item based on the result of the examination or the image diagnosis of the subject, and the predicted value is displayed on the display of the terminal device. At this time, since the predicted value is shown in the graph in the second change mode different from the normal mode and the first change mode, the examiner can reliably read the predicted value from the graph.

(5) A terminal device including:
a display displaying a result of an examination or an image diagnosis related to an eyeball of a subject;
a display controller configured to control a display content and a display mode; and
a terminal memory configured to store the result of the examination or the image diagnosis in association with device information including a manufacturer or a model name of an ophthalmic device used at that time,
wherein the display displays the result of the examination or the image diagnosis in a time-series graph, and
wherein in a case where a change in the manufacturer or the model name from that in a previous examination is detected based on the device information, the display controller changes the display mode of the graph.

Since the display of the terminal device displays the result of the examination or the

image diagnosis in a time-series graph, the examiner can confirm a transition of an examination numerical value or an image. For example, the manufacturer of the ophthalmic device in the examination may be changed from that used in the previous examination, but the result and the device information related to the ophthalmic device used at that time are stored in association with each other. Therefore, the display controller of the terminal device detects the change in the ophthalmic device based on the device information and changes the display mode of the graph. Accordingly, the examiner determines the presence or absence of an abnormality taking into consideration that the ophthalmic device is changed, and thus a current examination result can be correctly evaluated.

(6) The terminal device according to (5),
wherein the terminal memory stores a comparison table related to a numerical tendency for each examination item of each of the manufacturers, and
wherein the display controller causes a window including at least the comparison table to appear when any examination time in the graph is designated.

The terminal memory stores the comparison table related to the numerical tendency for each examination item of each manufacturer, and the examiner can confirm contents of the comparison table on the display. In particular, when an examination date in the graph is designated, the display controller causes the window to appear on the display and displays the comparison table, and thus the examiner can acquire device characteristics of a device that gives a higher numerical value, a device that gives a lower numerical value, or the like.

What is claimed is:

1. An ophthalmic system comprising:
a server including a memory storing a result of an examination or an image diagnosis related to an eyeball of a subject; and
a terminal device connected to the server, the terminal device including:
a display displaying the result of the examination or the image diagnosis; and
a display controller configured to control a display content and a display mode,
wherein the memory stores the result of the examination or the image diagnosis in association with device information including a manufacturer or a model name of an ophthalmic device used at that time,
wherein the display displays the result of the examination or the image diagnosis in a time-series graph, and
wherein in a case where a change in the manufacturer or the model name from that in a previous examination is detected based on the device information, the display controller sets the display mode of the graph to a first change mode different from a normal mode without the change.

2. The ophthalmic system according to claim 1,
wherein the memory stores a comparison table related to a numerical tendency for each examination item of each of the manufacturers, and
wherein the display controller causes a window including at least the comparison table to appear when any examination time in the graph is designated.

3. The ophthalmic system according to claim 2,
wherein in the window, device display is performed in which the manufacturer or the model name of the ophthalmic device used in the examination is recognizable.

4. The ophthalmic system according to claim 1, further comprising:
a machine learning model connected to the server and configured to be subjected to machine learning such that many results of the examination or the image diagnosis for each examination item are received as training data, and a predicted value for a corresponding one of the examination items is output,
wherein the server includes a calculator configured to refer to the machine learning model to calculate the predicted value of a predetermined one of the examination items based on the result of the examination or the image diagnosis of the subject, and
wherein the display controller sets the display mode of the graph to a second change mode different from the normal mode and the first change mode when displaying the predicted value of the predetermined examination item.

5. A terminal device comprising:
a display displaying a result of an examination or an image diagnosis related to an eyeball of a subject;
a display controller configured to control a display content and a display mode; and
a terminal memory configured to store the result of the examination or the image diagnosis in association with device information including a manufacturer or a model name of an ophthalmic device used at that time,
wherein the display displays the result of the examination or the image diagnosis in a time-series graph, and
wherein in a case where a change in the manufacturer or the model name from that in a previous examination is detected based on the device information, the display controller changes the display mode of the graph.

6. The terminal device according to claim 5,
wherein the terminal memory stores a comparison table related to a numerical tendency for each examination item of each of the manufacturers, and
wherein the display controller causes a window including at least the comparison table to appear when any examination time in the graph is designated.
